(19) United States
(12) Patent Application Publication
(10) Pub. No.: US 2022/0183935 A1
(43) Pub. Date: Jun. 16, 2022

(54) 3D BIOPRINTER USING PLANT STEM CELLS APPLIED TO SKIN TISSUE RECREATION FOR FACIAL REJUVENATION AND FACIAL BIOMASK FROM 3D BIOPRINTING

(71) Applicant: DERMYAH INOVACOES EM BELEZA E BEM ESTAR LTDA, Americana (BR)

(72) Inventor: Lucas PEREIRA, Americana (BR)

(21) Appl. No.: 17/548,737

(22) Filed: Dec. 13, 2021

(30) Foreign Application Priority Data
Dec. 11, 2020 (BR) 20 2020 025444 3

(51) Int. Cl. A61K 8/02; A61K 8/97; A61Q 19/08; B33Y 30/00; B33Y 50/00; B33Y 80/00; B29C 64/386; G06F 16/532

(52) U.S. Cl. CPC A61K 8/0212; A61K 8/97; A61Q 19/08; B33Y 30/00; B29L 2031/753; B33Y 80/00; B29C 64/386; G06F 16/532; B33Y 50/00

(57) ABSTRACT

A 3D (three dimensional) Bioprinter that uses plant stem cells applied to the recreation of skin tissue for facial rejuvenation, in addition to the facial biomask produced from the 3D bioprinter. The field of application of this utility model patent application is related to the Esthetics and Cosmetology industries. 3D bioprinting with plant stem cells is the latest and most revolutionary treatment for the prevention of aging and facial rejuvenation. It is a one hundred percent natural protocol for recreating the beauty of human skin.

### FIELD OF APPLICATION

The present utility model patent application refers to a 3D (three dimensional) bioprinter that uses plant stem cells applied to the recreation of skin tissue for facial rejuvenation, in addition to the facial biomask produced from the 3D bioprinter. The field of application of this utility model patent application is related to the Esthetics and Cosmetology industries.

### DESCRIPTION OF THE PRIOR ART

Three-dimensional (3D) printing is a fabrication method of solid objects from a digital file containing spatial coordinate information. The creation of a 3D printed prototype is achieved by depositing successive layers of material until the object is completed. An important feature of this methodology is that various types of materials can be used, such as metals, resins, polymers and more recently biological components, including biomolecules and cells. When biological components are involved in the fabrication process, the process is called 3D bioprinting.

This technique constitutes one of the most recent innovations in biotechnology and synthetic biology, enabling from the production of small, organized groups of cells used for activity assays to the production of whole organs for transplantation. As far as assays are concerned, 3D bioprinting provides more interesting models than traditional cell cultures, in which the cells are disorganized and out of their real environment. The use of three-dimensional printing makes it possible to recreate more closely the context in which the cells are found in the body, better preserving its function. Therefore, biological activity tests become more precise and realistic, highlighting effects that substances may have in the body that are were not detected in previous tests.

Bioprinting can be defined as the use of cells and other biological products in stack printing for the assembly of tissues and organs from computer-aided deposition of layers, which can be used in regenerative medicine, pharmacokinetic studies as well as other biological studies (Guillemot et al. 2010). This recent technology can be classified according to its operating principle, being: bioprinting based on inkjet, extrusion and laser printing (Tong et al. 2012). From the modification of commercial printers, a printing mechanism was developed using inkjets, which can be made as a continuous jet or drop by drop. The former produces an uninterrupted trace, while the latter deposits controlled ink droplets at precisely coordinated positions, printing in modular droplet units. Only drop-on-demand ink printing is considered suitable for bioprinting, however the continuous inkjet method requires a conductive ink. A cartridge is loaded with bio-hydrogel cells and is printed in well-placed droplets that are generated from a printhead by the close control of a thermal or piezoelectric actuator (equipment that can be used in building flexible and customized structures). The thermal system is most prevalent for cell printing, and although the actuator temperature can exceed up to 200° C., the cells experience only small temporary temperature changes during the process.

Currently, inkjet printheads tend to use the Micro-Electro Mechanical system, which enables printing of highly viscous materials (Irvine & Venkatraman 2016). It is worth commenting that, inkjet bioprinting, prints living cells in droplet form through cartridges, and it is one of the most promising technologies that allow the biofabrication of tissues or organs (Chang et al. 2008). A major advantage of this technique is that it allows printing of individual cells as well as cell aggregates by controlling the concentration, resolution, volume decrease and diameter of the printed cells (Ozbolat & Yin Yu 2013). This technology uses living cells and aims at the exact deposition of encapsulated cells into 3D-structured cylindrical filaments using biopolymers such as chitosan, ceramics, and other biocompatible materials (Ozbolat & Yin Yu 2013).

Extrusion bioprinting is the most promising technique described today, since it allows the fabrication of organized constructs of clinically relevant sizes within real time. This technique is able to produce a continuous trace of the bioink filament (a substance made of living cells that can be printed in a programmed format) from a syringe, similar to the toothpaste mechanism that emerges from a squeezed tube. The hydrogel bioink is forced from the syringe using pneumatic air pressure; the syringe is attached to the printing channel moving the z-y direction on a collector that moves the x-axis, so the hydrogel can be deposited in 3D patterns and this direction arrangement can vary between bioprinter models. Numerous biocompatible hydrogel polymers can be used as bioinks for extrusion bioprinting, it is worth commenting that in this process the bioink material must be selected according to the type of cell to be used for bioprinting and have the ability to provide a relatively high cell concentration. The mechanical screw plunger promotes better control over the flow of bioink, which is important for improving standardization (Pati et al. 2015, Maher et al. 2009, Campos et al. 2012). However, for stem cell printing, mechanical and/or pneumatic screw extrusion can produce large pressure drops across the printer nozzle. It is worth highlighting that pressure drop is associated to deformation and apoptosis of the bioink encapsulated cells (Irvine & Venkatraman 2016).

The use of the laser bioprinting technique was developed from direct etching and laser-induced transfer technologies. This method works by focused laser stimulation from the top surface of an apparent layer that absorbs energy. A cell containing a biopolymer or bioink is coated onto the bottom surface of the energy absorbing layer, laser stimulation vaporizes the surface material, generating a pressure bubble that propels a drop of bioink to a collection/receiving substrate for placement of the cell layer. Contrary to inkjet printing, laser-assisted bioprinting does not require a dispensing nozzle, and this reduces the shear stress experienced by the stem cells during deposition and also eliminates the occurrence of printer nozzle clogging. It can be considered that this method has good cell printing properties, since it is able to use viscous biofilters and prints high cell densities with good post-deposition feasibility. Furthermore, the resolution can be approximately 10 μm, but this technique is considered the most expensive and complex, requiring applied research on defined cell types such as: bone, cartilage and connective tissue, among others (Griffin et al. 2015, Irvine & Venkatraman 2016).

Bioprinting allows precise placement of biological products for the purpose of reconstituting tissue biology. The technology allows the development of organ or tissue constructs that do not require substantial vascularization, as well as mini-tissue and/or mini-organ models mimicking natural biology for studies of new drugs, such as for the treatment of cancer (Ozbolat 2015, Tong et al. 2012, Mutreja et al. 2015). Bioprinting is a technique that can be used to treat cranioplasty, since doctors can develop prototypes to study the deformity, as well as evaluate and train the surgical technique before the procedure on humans.

It is emphasized that this procedure can decrease surgical risks, because the surgeon can study the pathology of the individual in a unique and specific way (Lee et al. 2009). It is also possible to evaluate the data produced from 3D prints and based on statistical data of feasibility in using this technique, to observe and evaluate body structures obtained from scanned images obtained by means of computed tomography (CT) of 3D tissue models. Therefore, it can be said that based on these 3D models, bio-models (physical models), 3D printed bone and cartilage structures can be important tools for the development of new high-precision surgical techniques for implants and rapid prototyping (Choonara et al. 2016). Bioprinting models can be used for preoperative preparation as a mold to establish implantable devices, for example, cartilage and bone tissue to produce a biological mold according to each the need of each patient, thus reducing the probability of rejection (Choonara et al. 2016).

Due to its major advantage in forming various cell types, there has been rapid development in making whole functional tissues and organs in 3D, such as heart, lung, liver and kidneys (Ozbolat 2015). Research on the production of organs and mini-organs has increased, generating expectations of their use in tissue grafts and organ transplantation. However, tissue engineering strategy cannot yet enable the fabrication of fully functional tissues or organs so far (Ozbolat 2015).

Tissue engineering is an emerging field in which materials science and cell biology contribute to make new tools possible, such as the implantation of biofabricated tissues in regenerative medicine. The ultimate goal of bioprinting is to recapitulate the natural process of tissue formation by cells to assemble synthetic frameworks that are able to mimic the natural tissue microenvironment.

The core of this multidisciplinary challenge is to understand how cell behavior is regulated through cell/time interactions, and to reproduce this knowledge to produce biomaterials compatible with the production of tissues, cells and organs (Tong et al. 2012).

Bioprinting of tissues, organs, and cellular frameworks can be carried out with current 3D printing techniques, but they require the material of choice for bioprinting to be a fluid or powder, as the structure of the material must be rapidly adjusted in order for stability to maintain the integrity of the print. However, traditionally this requires a material that can be printed at high temperature for metals and polymers or even materials that can be dissolved in volatile solvents. The cells can be included and printed on a cytocompatible material, so the subsequent construct will be immediately formed with a consistent and highly viable cell distribution throughout the entire bioprinting process. Furthermore, the distribution and formation of different types of cells can potentially be placed and guided to form the desired structure, being cellular and connective tissue, and in the future this technique will also enable the development and 3D printing of organs (Irvine & Venkatraman 2016).

Bioprinting may be the technology of the future, however, aspects such as hierarchical arrangement of cells or the construction of tissue blocks in a 3D microenvironment and factors such as the post-bioprinting maturation phase are as important as the printing process itself. Another important component in this scenario is the choice of biomaterial, as it will provide all the biochemical (chemokines, growth factors, adhesion factors, or signaling proteins) and physical (interstitial flow, mechanical and structural properties of the extracellular matrix) aspects. These factors promote an environment with higher viability for cell survival, aiding cell motility and differentiation. Furthermore, the biomaterial must have high stability in the mechanical and structural integrity after bioprinting, allowing the differentiation of autologous stem cell lines into tissue-specific cell lines, thus facilitating endogenous tissue grafting without generating an immune response (Ozbolat 2015, Mutreja et al. 2015). Stem cells have great differentiation potential, with the capacity for proliferation and self-renewal, giving origin to different specialized cell lineages (Pereira 2008). There are several classes of stem cells, which can be classified into totipotent, pluripotent and multipotent stem cells; and according to their origin they can be embryonic or adult (Vogel 2000, Souza et al. 2003, Silva Júnior et al. 2009). Stem cells are defined by having a self-renewal and multilineage potential, i.e., they can be stimulated to form many different types of functional cells. There are three main types of stem cells commonly used in bioengineering: mesenchymal, embryonic and induced pluripotent. Mesenchymal stem cells are also referred to as mesenchymal stromal cells or multipotent cells, and these cells have the ability to differentiate into osteoblasts, adipocytes or chondroblasts in vitro. Additionally, they also have the ability to become other types of mesenchymal and non mesenchymal cells such as myocytes, ligament cells, smooth muscle cells, endothelial cells, cardiomyocytes, hepatocytes and neural cells (Irvine & Venkatraman 2016). Mesenchymal stem cells were originally isolated from bone marrow, but have since been located in many tissues such as: liver, lung, muscle, adipose, amniotic fluid/Wharton's jelly, umbilical cord, and placenta. The particular source of mesenchymal stem cells often confers advantageous aspects compared to other sources, since, for example, the concentration of stem cells harvested from adipose tissue is significantly higher than from other sources (Lindroos et al. 2011, Irvine & Venkatraman 2016).

3D bioprinting with plant stem cells is the latest and most revolutionary treatment for the prevention of aging and facial rejuvenation. It is a one hundred percent natural protocol for recreating the beauty of human skin. From the bioprinting of a living skin, one hundred percent compatible with the human body, the main factors of aging can be combated:
cellular aging (telomore shortening);
oxidative stress;
loss of transepidermal water.

Therefore, the purpose of the present utility model patent application refers to a 3D (three dimensional) bioprinter that uses plant stem cells applied to the recreation of skin tissue for facial rejuvenation, in addition to the facial biomask produced from the 3D bioprinter.

The advantages of using a 3D Bioprinter with stem cells are numerous The following can be highlighted:
one hundred percent natural, non-invasive treatment that can be performed by any type of skin and at any time of the year;
without contraindications, and can be performed even by pregnant women;
green tea stem cells with high renewal power;
using nanotechnology, it is possible to achieve a skin penetration power of up to 100 times more than any cream or cosmetic product available in the market;
antioxidant and anti-inflammatory action: prevents aging at cellular level (telomere shortening and cell death);
protects against ultraviolet radiation (sunlight), the main factor in skin aging;
improves the blood microcirculation of the dermis, improving the availability of oxygen and nutrients to the skin;
protective effect on vitamin C (essential for the formation of collagen).

In addition to plant stem cells, the face mask has more than 15 bioactives: hyaluronic acid; vitamin C; vitamin B complex; amino acids; fatty acid complexes; polyphenols and catechins from green tea; seaweed.

### DESCRIPTION OF THE FIGURES

FIGS. 1A, 1B and 1C represent an overview of the 3D bioprinter.

FIGS. 3A and 3B show the fabrication of the biomask and the application of the biomass on the face.

### DETAILED DESCRIPTION OF THE INVENTION

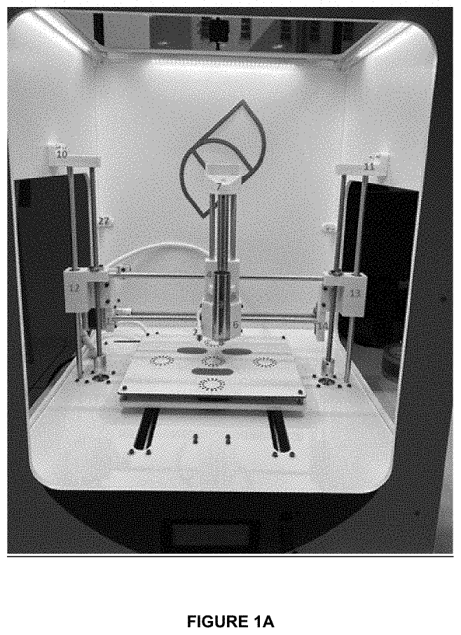

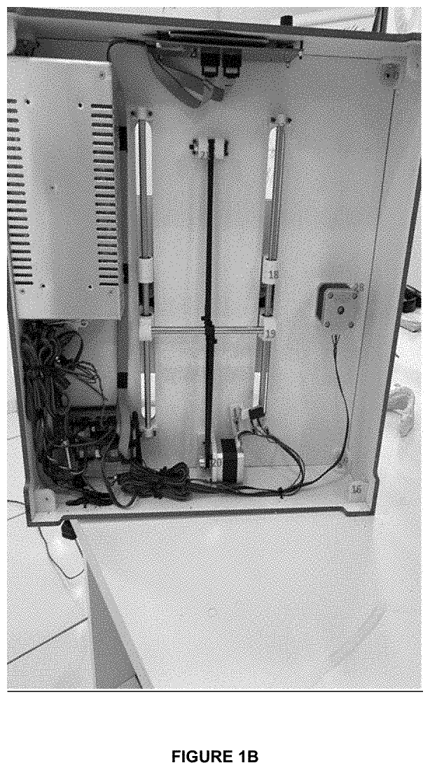

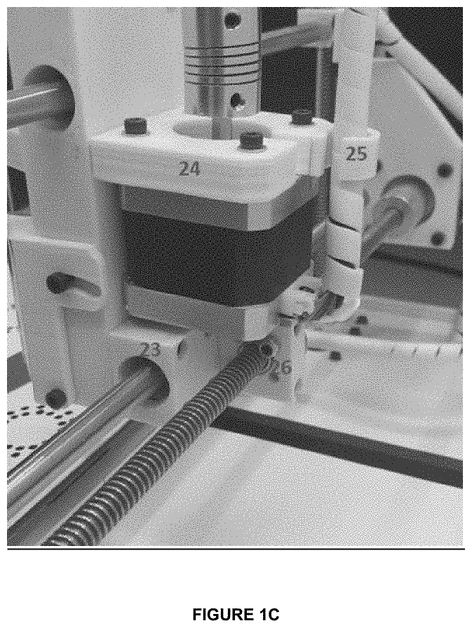

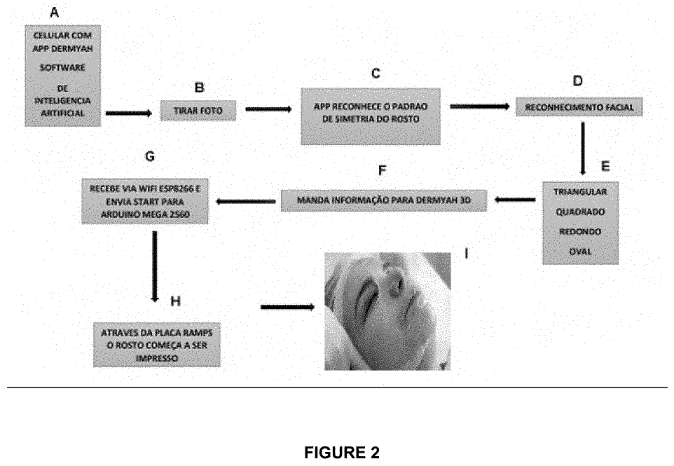

FIGS. 1A, 1B and 1C represent an overview of the 3D bioprinter, showing: (1) X-axis Endstop Arm; (2) Z-axis Endstop Arm; (3) Stainless Steel Plunger; (4) head part 1; (5) head part 5; (6) head part 4; (7) head part 6; (8) stainless steel cap; (9) stainless steel tube; (10) Left Z-axis Support; (11) Left Z-axis Support; (12) Right Side 1; (13) Left Side 1; (14) Left Side 1. 1; (15) Right Side 1. 1; (16) Support Bracket; (17) Y-axis Endstop Support; (18) Y-axis Table Bearing Support; (19) Y-axis Table Bearing Support with Belt Attachment; (20) Y-axis Motor Base; (21) Y-axis Pulley Support; (22) Y-axis Support; (23) Head Part 3; (24) Head Part 2; (25) Cable Support; (26) Head Part 7; (27) Middle Bracket; (28) Right and Left Z-axis Bottom Support.

FIG. 2 represents the flowchart for 3D bioprinter use. A mobile phone with the artificial intelligence application (2A) takes a photo (2B) and sends it to an application (2C) with software used in the application that is developed by java language, where the face is scanned using a photo taken on the spot. The photo is sent to a database with an algorithm that recognizes the shape of the face, being a symmetry pattern of the face and promotes facial recognition (3D), which can be of geometries (3E): triangular, square, round, oval. The application (3C) sends the information to the 3D Bioprinter (1). After the reading and recognition is performed, the information is sent via wifi to the 3D bioprinter (3F). The software and hardware used in the 3D bioprinter features an ESP8266 wifi receiver, which is a microcontroller that includes Wi-Fi communication capability (Arduino mega 2560 together with RAMPS 1.4) (3G). Through the RAMPS board (3H), these are responsible for converting the information received into a customized biomask (3I).

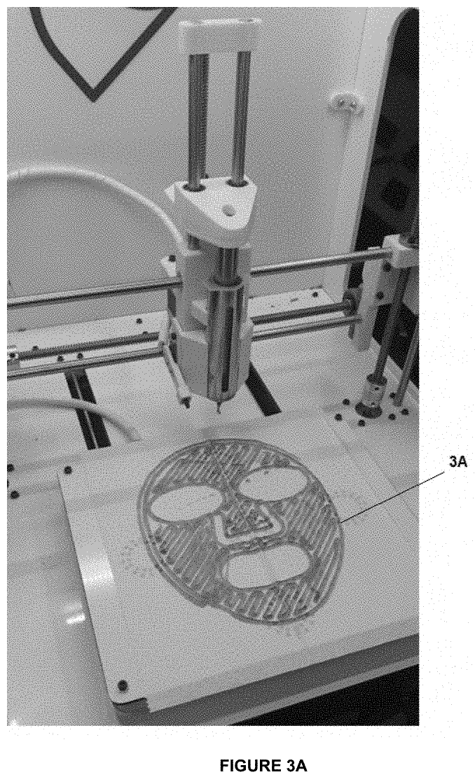

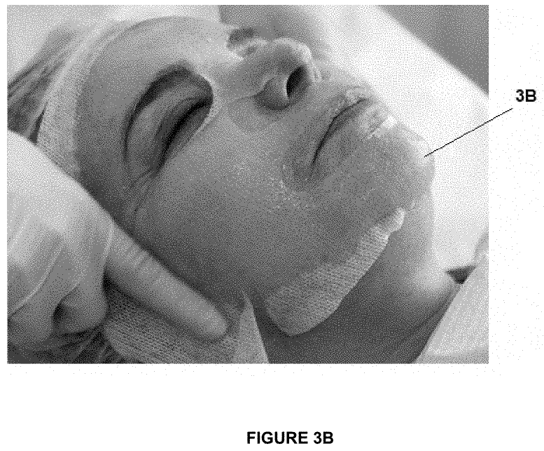

FIGS. 3A and 3B show: the fabrication of the biomask (3A) and the application of the biomass (3A) on the face (3B).

### 10/10

The 3D Bioprinter allows cellular bioink of plant origin to be printed in 3D geometry for application in cosmiatry and advanced esthetics.

The 3D Bioprinter also has an exclusive Artificial Intelligence system that makes a facial scanner installed in the mobile phone of the professional work together with the bioprinter, recognizing patterns of face shapes and creating a mask, tailored to the patient.

The cells are extracted through an exclusive fermentation and conversion process of the actives in Bionanotechnological format, thus being possible that the printed cells after being placed on the skin and through occlusion penetrate to the last layer of the skin (Basal layer), thereby having an increase in the germ stem cells of the patient.

1) A 3D bioprinter comprising: X-axis Endstop Arm; -axis Endstop Arm; Stainless Steel Plunger; head part 1; head part 5; head part 4; head part 6; stainless steel cap; stainless steel tube; Left Z-axis Support; Left Z-axis Support; Right Side 1; Left Side 1; Left Side 1. 1; Right Side 1. 1; Support Bracket; Y-axis Endstop Support; Y-axis Table Bearing Support; Y-axis Table Bearing Support with Belt Attachment; Y-axis Motor Base; Y-axis Pulley Support; Y-axis Support; Head Part 3; Head Part 2; Cable Support; Head Part 7; Middle Bracket; Right and Left Z-axis Bottom Support.

2) The 3D bioprinter according to claim 1, comprising the following operating stages:
a mobile phone with the artificial intelligence application takes a photo and sends it to an application with software used in the application that is developed by java language, where the face is scanned through a photo taken on the spot; where the photo is sent to a database with an algorithm that recognizes the shape of the face, being a pattern of symmetry of the face and promotes face recognition, which can be of geometries: triangular, square, round, oval; where said application sends the information to the 3D Bioprinter; where, after the reading and recognition is performed, the information is sent via wifi to the 3D bioprinter; where said software and hardware used in the 3D bioprinter has an ESP8266 wifi receiver, which is a microcontroller that includes wifi communication capability (Arduino mega 2560 together with RAMPS 1.4); where, through the RAMPS board, these are responsible for converting the information received into a customized biomask.

3) The 3D bioprinter according to claim 1, containing stem cells from green tea.

4) The 3D bioprinter according to claim 1, recreating a skin tissue for facial skin tissue rejuvenation.

5) A facial biomask processed by 3D bioprinter.

6) The facial biomask according to claim 5, wherein the cells used in its fabrication are extracted through an exclusive fermentation and conversion process of the actives in Bionanotechnological format.

7) The facial biomask according to claim 5, wherein printed cells after being placed on the skin and through occlusion, penetrate to the last layer of the skin (Basal layer), thereby having an increase in the germ stem cells of the patient.
